(19) United States
(12) Patent Application Publication
(10) Pub. No.: US 2022/0234742 A1
(43) Pub. Date: Jul. 28, 2022

(54) Aircraft Sanitizer

(71) Applicant: Antonio Alvarez, Miami, FL (US)

(72) Inventor: Antonio Alvarez, Miami, FL (US)

(21) Appl. No.: 17/160,279

(22) Filed: Jan. 27, 2021

(51) Int. Cl. B64D 13/06; A61L 9/20

(52) U.S. Cl. CPC B64D 13/06; B64D 2013/0655; A61L 9/20

(57) ABSTRACT

A complete passenger service unit (PSU) that includes a ducted housing that cools a stepped-down transformer from native 28 V DC aircraft power to 12-36 V DC to power and ultraviolet light producing ultraviolet wavelengths between about 200 nm and 235 nm. The ventilation air provided to all PSU stations is fed into the ducted housing to eliminate the need for additional fans or other cooling means for the transformer.

### CROSS-REFERENCES TO RELATED APPLICATIONS

None.

### STATEMENT REGARDING FEDERAL SPONSORED RESEARCH OR DEVELOPMENT

None.

### NAMES OF PARTIES TO A JOINT RESEARCH AGREEMENT

None.

### REFERENCE TO A “SEQUENCE LISTING”, A TABLE, OR A COMPUTER PROGRAM LISTING APPENDIX SUBMITTED ON COMPACT DISC AND INCORPORATION-BY-REFERENCE OF THE MATERIAL ON THE COMPACT DISCLOSURE

None.

### STATEMENT REGARDING PRIOR DISCLOSURES BY AN INVENTOR OR JOINT INVENTOR

None.

### BACKGROUND OF THE INVENTION

### 1. Field of the Invention

The present invention relates to sanitizing commercial aircraft, and more particularly, to an improved device and method of use of a passenger service unit (PSU) that adapts ship power to project ultraviolet light onto a interior of an aircraft while providing cooling to the power supply.

### 2. Description of Related Art Including Information Disclosed Under 37 CFR 1.97 and 37 CFR 1.98

Several designs for passenger service units have been designed in the past. However, none of them include a means to supply the necessary power for specified wavelengths of ultraviolet (UV) coupled to the onboard ventilation system to provide cooling without a fan in the passenger service unit.

Applicant believes that the closest reference corresponds to widely available passenger service units in the industry that provide visible light as well as ventilation from the aircraft. Other solutions for sanitizing aircraft include a chemical fog that lands on surfaces. These chemical fogs produce residue and may damage aircraft upholstery and interior parts through corrosion. Fogs also have the risk of inhalation by personnel using the device.

Other ultraviolet solutions have used ineffective wavelengths of light. The available UV devices have a potential to damage human skin as well as potentially degrading upholstery. These other solutions also fail in that they require a cooling solution for the heat generated by the power supply. Each of these problems has been solved by the present invention.

Other patents describing the closest subject matter provide for a number of more or less complicated features that fail to solve the problem in an efficient and economical way. None of these patents suggest the novel features of the present invention.

A brief abstract of the technical disclosure in the specification and title are provided as well for the purposes of complying with 37 CFR 1.72 and are not intended to be used for interpreting or limiting the scope of the claims.

Without limiting the scope of the invention, a brief summary of some of the claimed embodiments of the invention is set forth below. Additional details of the summarized embodiments of the invention and/or additional embodiments of the invention may be found in the detailed description of the invention below.

### BRIEF SUMMARY OF THE INVENTION

It is one of the main objects of the present invention to provide a direct physical replacement for existing passenger service units and aircraft.

It is another object of this invention to provide a device that is safe for both the occupant of the aircraft and the cleaning crew administering the sterilization procedure.

It is still another object of the present invention to provide a quiet device that uses minimal moving parts for a low cost and easy replacement of existing passenger service units during installation.

It is yet another object of this invention to provide such a device that is inexpensive to manufacture and maintain while retaining its effectiveness.

Further objects of the invention will be brought out in the following part of the specification, wherein detailed description is for the purpose of fully disclosing the invention without placing limitations thereon.

These and other embodiments which characterize the invention are pointed out with particularity in the claims annexed hereto and form a part hereof. However, for a better understanding of the invention, its advantages and objectives obtained by its use, reference can be made to the drawings which form a further part hereof and the accompanying descriptive matter, in which there are illustrated and described various embodiments of the invention.

### BRIEF DESCRIPTION OF THE SEVERAL VIEWS OF THE DRAWING

With the above and other related objects in view, the invention exists in the details of construction and combination of parts as will be more fully understood from the following description, when read in conjunction with the accompanying drawings in which:

FIG. 1 shows an elevation cross-section of an interior cabin of a typical passenger jet with the present device installed.

FIG. 2 shows a close-up elevation cross-section of an individual passenger service unit.

FIG. 3 shows a chart identifying particular wavelengths of sterilizing light that may be used with the current system.

### DETAILED DESCRIPTION OF THE INVENTION

While this invention may be embodied in many different forms, there are described in detail herein specific embodiments of the invention. This description is exemplary of the principles of the invention and is not intended to limit the invention to the particular embodiments illustrated and described.

For the purpose of this disclosure, like reference numerals in the figures shall refer to like features unless otherwise indicated or is obvious by context.

The subject device and method of use is sometimes referred to as the device, the invention, the passenger service unit, the PSU, the overhead panel, the ventilation system, the sterilization system, the machine or other similar terms. These terms may be used interchangeably as context requires and from use the intent becomes apparent. The masculine can sometimes refer to the feminine and neuter and vice versa. The plural may include the singular and singular the plural as appropriate from a fair and reasonable interpretation in the situation.

Referring now to the drawings, where the present invention is generally referred to with numeral 10, it can be observed that it basically includes a passenger service unit (PSU) 12, a seat 14, a vent 16, a light 18, a power supply 20, an air supply 22, a conduit 24, a connector 26, a ceiling panel 28, a ducted housing 30, a stepdown converter circuit 31, transformer 32, a connector 34, an oxygen mask 36, controls 38, a connector 40, a processor 42, a button 44 and a housing 46.

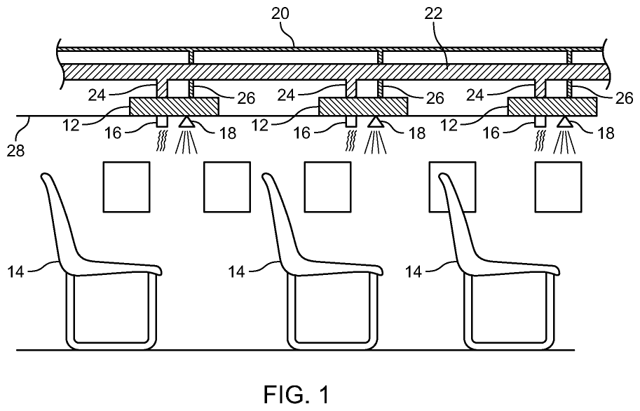

FIG. 1 generally shows a cross-section of an interior of a commercial passenger aircraft. Several rows of seats 14 are shown adjacent to windows in the side wall of the aircraft. Each seat or row of seats has an overhead passenger service unit (PSU).

Commercially available PSU's commonly include a fresh air vent, a reading light, a call button, an informational light or other features depending on the make and model of the PSU and the carriers needs. Many PSUs also contain a deployable oxygen mask that drops from a panel in the PSU if supplemental oxygen is needed by the passengers.

Generally, the light provided by the prior art reading light is a visible light allowing the passenger to have additional illumination to read or conduct other activities while traveling. Earlier attempts to install an ultraviolet (UV) emitter have encountered problems with overheating, unsafe wavelengths of light and power supply issues, among other problems.

Many modern commercial aircraft use a nominally 28 V direct current (DC) electrical system to provide power for the other features in each individual PSU. Attempts to integrate and ultraviolet sterilizing light have been limited to ultraviolet lamps producing approximately 254 nm wavelength light. Ultraviolet emitters using safer wavelengths are not available in a 28 VDC configuration. Some attempts at providing a transformer have failed due to the excess heat generated by stepping down the voltage.

Contemporary medical literature suggests that use of 254 nm light adversely affects the skin and retina of humans when exposed. The longer a person is exposed to such wavelengths, the greater the risk of damage. However, it is the same damage that breakdowns the DNA and RNA of a virus. Therefore, the 254 nm class of UV light has been limited to use when people are not present to absorb these harmful wavelengths.

A solution was needed to continually sterilize the interior of an aircraft while occupied by travelers. As passengers are in the cabin airborne particles may be transmitted between passengers. By having a suitable sterilizing ultraviolet light available during the use of an aircraft, the airborne viruses, germs and bacteria may be reduced or eliminated while the cabin is occupied and the potentially contaminated air is being inhaled by crew and passengers.

Additionally, with a safer wavelength, the human friendly ultraviolet light may be used continually at a lower dose. This both makes the exposure to ultraviolet light safer for the passengers and crew, but also may more thoroughly disinfect not only the air but all interior surfaces of the aircraft.

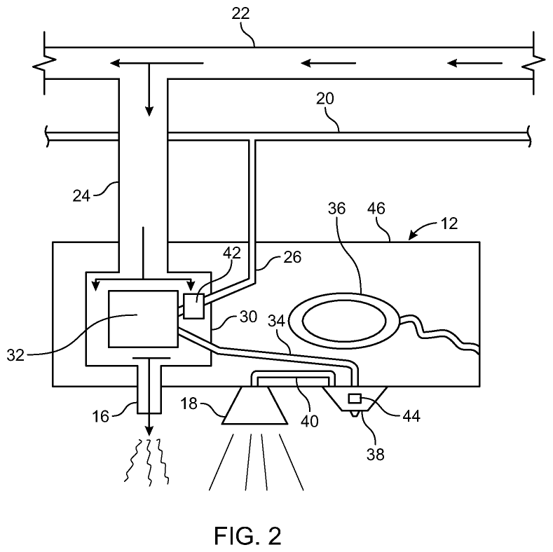

Looking at FIGS. 1 and 2 in combination, a design for an improved passenger service unit (PSU) is shown. The present PSU is preferably a direct, slide in replacement for current PSUs in use. This makes an economical and easy replacement installation. The existing ventilation supplies air through the air supply 22 as well as the power supply 20 used for prior art versions of PSUs remain suitable for the present design.

The ship's power supply is provided by the wired power supply 20 with a connector 26 attaching to each PSU individually or for a multiple seat PSU over a row of seats. Similarly, the fresh air supply delivered to prior art PSU's is delivered down the conduit air supply 22 and connected to each individual PSU with a conduit 24. Oxygen provided to the oxygen masks 36 is similarly used analogous to current art systems.

A transformer 32 is provided inside the ducted housing 30 of the PSU 12. Instead of cooling fins or a dedicated cooling fan for the transformer 32 inside each PSU, the ship's vent air provided through air supply 22 and conduit 24 is ducted over and around the transformer 32 as the vent air passes through the ducted housing 30 inside the PSU 12. Therefore, no additional electrical connections relating to providing cooling functionality to the transformer 32 are necessary to be included in the PSU. This reduces the complexity, costs and need to retrofit existing systems within the aircraft.

The ducted housing 30 contains at least a portion of the transformer 32. The purpose of the ducted housing 30 is to ensure the vent air delivered from the air supply 22 on its way to the passenger vent 16 is available to cool the transformer 32. Thus, with no moving parts, the transformer 32 may be adequately cooled to maintain safe operational temperatures.

The transformer 32 is provided to convert the 12-36 V ship's power to the 12 V DC power suitable for use with the 222 nanometer ultraviolet light 18. The light 18 is preferably a low power consumption light generator, such as a light emitting diode (LED). However, other lights capable of emanating the appropriate wavelength of light may be suitable. It should be appreciated that different aircraft and aircraft systems may typically supply between 12-36 V DC power and the components in the present system should be adapted to the particular installation. Some aircraft also use 110 VAC or other AC voltages and appropriate transformers may be specified to achieve the design established within the inventive concepts.

Generally, wavelength less than 200 nm can produce ozone. Ozone being generated into the atmosphere near the passengers may be inhaled and may come into contact with the respiratory system of those passengers. Ozone can aggressively attack on tissue by reacting chemically. Also when ozone is present, there are frequently other harmful pollutants created by the same processes that make ozone.

Ultraviolet light in the range of about 250 nm may cause damage to the ocular retina and skin of those exposed to the ultraviolet light long-term. If the present system is used with ultraviolet light having a wavelength greater than about 250 nm it is preferred to include a timer or to limit the time that the light is emitting to minimize the contact with humans. For example, after the passengers have deplaned, the light may be operated to sterilize the empty cabin.

The preferred wavelength for the least damage to human tissue is approximately 207 nm through 235 nm wavelength ultraviolet light. Within this range there has been shown to be minimal to no tissue damage from exposure to the degree passengers would experience on a commercial flight. In this range, the sanitizing light can remain on during the duration of a flight to sanitize and sterilize both the surfaces and the air to minimize contamination of the passengers.

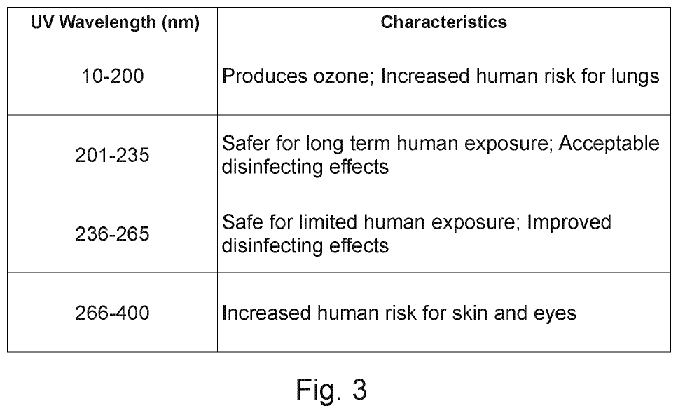

FIG. 3 shows a chart with the characteristics of the segments of the ultraviolet wavelengths from about 10 nm to 400 nm. The lower wavelengths, approximately 200 nm and below tend to produce ozone in levels that increase the risk to damage of the respiratory system. The ultraviolet wavelengths between about 201 nm and 235 nm are the safest for longer-term of human exposure while retaining acceptable disinfecting effects. This is the range preferred to be admitted by the light in the present design. Ultraviolet wavelengths from about 236 nm to 265 nm have the ability to more rapidly destruct viruses, bacteria and other pathogens but some studies show that exposure in these wavelengths should be limited. However, the time spent in an aircraft by a typical commercial traveler has not been found to be unsafe. Wavelengths over 266 nm are highly effective at deactivating pathogens but also have a slightly elevated risk of injury to the human skin and eyes after long-term exposure.

It should be appreciated that ultraviolet wavelengths over about 235 nm are well-suited to disinfect the interior of an aircraft while it remains unoccupied. This may be suitable for disinfecting the cabin between flights when it no passengers or staff are inside the aircraft. This allows for a rapid disinfecting of surfaces inside the aircraft.

In at least one version of the inventive concept, controls 38 are provided on an exterior surface of the PSU. The controls 38 can start a pre-programmed timer operated by the processor 42. Alternatively, the processor 42 may be able to detect the presence of a person in the seat below to stop or limit the ultraviolet exposure to that occupant.

A connector 26 connects the ships power supply 20 to the transformer 32. The stepped-down power is fed into the transformer 32 by the connector 34 into the processor 42. The processor 42 may operate the light 18 automatically. Alternatively, the controls 38 may manually connect and disconnect the power supplied to the light 18 by connector 40.

The stepdown converter circuit 31 inside the housing 30 is also shown on FIG. 2. The stepdown converter circuit 31 reduces the voltage from, for example 28 V, to 12, 24 or 36 V that is compatible with the transformer 32. The transformer 32 may also be characterized as an inverter.

An important version of the invention can be fairly described as a passenger service unit (PSU) installed in the ceiling over seats in a commercial aircraft comprised of, among other features, a transformer, a ducted housing and a light. The PSU is generally installed over an individual seat or row of seats in a commercial aircraft. Native to the aircraft is an air supply providing a fresh air source and electrical power. This air supply and power are provided to the PSU. The transformer is at least partially inside the ducted housing. The ducted housing is connected to the air supply so that the fresh air enters the PSU and passes through the ducted housing that contains the transformer. This fresh air cools the transformer to an acceptable temperature without the use of a fan or other cooling mechanism inside the PSU. The fresh air then exhausts into the ducted housing without commingling or otherwise affecting the cool, fresh air available to the passenger from the PSU. Alternatively, the air supply may be vented away from the passenger if the passenger does not want to have the air directed to their seats. The transformer is electrically connected to the power supply of the aircraft. Generally, the power from the aircraft is 28 V but the light is operable at 12-36 V, depending on the nature of the light. The power supply outputs the appropriate direct current voltage to the light. The light emits an electromagnetic radiation in a wavelength between about 200 nm and 265 nm inside the aircraft. In a related version of the device the wavelength of electromagnetic radiation, or light, is preferably between about 207 nm and 235 nm. The electromagnetic radiation is essentially an ultraviolet light fixture in the specified wavelengths. A preferred version of the device is dimensioned to fit a pre-existing PSU so the installation is a slide in replacement.

The foregoing description conveys the best understanding of the objectives and advantages of the present invention. Different embodiments may be made of the inventive concept of this invention. It is to be understood that all matter disclosed herein is to be interpreted merely as illustrative, and not in a limiting sense.

I/We claim:

1. A passenger service unit (PSU) comprised of a transformer, a ducted housing and a light;
the PSU is installed into an aircraft;
the aircraft has an air supply and power provided to the PSU;
the ducted housing contains the transformer;
the ducted housing is connected to the air supply;
the air supply flows into the ducted housing to cool the transformer and then exhausts the air supply into the ducted housing;
the transformer is electrically connected to the power supply and to the light;
the light emits an electromagnetic radiation wavelength between 200 nm and 265 nm inside the aircraft.

2. The passenger service unit of claim 1 further characterized in that the emitted electromagnetic radiation wavelength between 207 nm and 235 nm inside the aircraft.

3. The passenger service unit of claim 1 further characterized in that dimensions of the PSU are adapted to be a slide in replacement for a pre-existing PSU.
